(19) United States
(12) Patent Application Publication
(10) Pub. No.: US 2016/0238601 A1
(43) Pub. Date: Aug. 18, 2016

(54) METHODS AND COMPOSITIONS FOR CORONAVIRUS DIAGNOSTICS AND THERAPEUTICS

(71) Applicant: THE UNIVERSITY OF NORTH CAROLINA AT CHAPEL HILL, Chapel Hill, NC (US)

(72) Inventors: Ralph Baric, Haw River, NC (US); Sudhakar Agnihothram, Chapel Hill, NC (US); Boyd Yount, Hillsborough, NC (US)

(21) Appl. No.: 15/029,190

(22) PCT Filed: Oct. 14, 2014
(86) PCT No.: PCT/US2014/060429

§ 371 Date: Apr. 13, 2016

Related U.S. Application Data
(60) Provisional application No. 61/890,804, filed on Oct. 14, 2013.

(51) Int. Cl. G01N 33/569; C12N 7/00; A61K 39/215

(52) U.S. Cl. CPC G01N 33/56983; A61K 39/215; C12N 7/00; G01N 2333/165; G01N 2469/10; G01N 2500/20; G01N 2500/10; C12N 2770/20034; A61K 2039/575

(57) ABSTRACT

The present invention provides methods and compositions for detecting a coronavirus in a sample and identifying the subgroup of the coronavirus in the sample.

### STATEMENT OF PRIORITY

This application is a 35 U.S.C. §371 national phase application of International Application Serial No. PCT/US2014/060429, filed Oct. 14, 2014, which claims the benefit, under 35 U.S.C. §119(e), of U.S. Provisional Application Ser. No. 61/890,804, filed Oct. 14, 2013, the entire contents of each of which are incorporated by reference herein.

### STATEMENT OF FEDERAL SUPPORT

This invention was made with government support under Grant Nos. AI085524, AI057157, and U19 AI107810 awarded by the National Institutes of Health. The government has certain rights in the invention.

### FIELD OF THE INVENTION

The present invention relates to methods and compositions for detecting and identifying a coronavirus by subgroup as well as treating and/or preventing a disease or disorder caused by a coronavirus infection.

### BACKGROUND OF THE INVENTION

Updated approaches are needed to rapidly respond to new emerging diseases, especially early in the epidemic when prompt public health intervention strategies can limit mortality and epidemic spread. In particular, emerging respiratory coronaviruses offer a considerable threat to the health of global populations and the economy. Coronaviruses (CoVs) constitute a group of phylogenetically diverse enveloped viruses that encode the largest plus strand RNA genomes and replicate efficiently in most mammals. Human CoV (HCoVs-229E, OC43, NL63, and HKU1) infections typically result in mild to severe upper and lower respiratory tract disease. Severe Acute Respiratory Syndrome Coronavirus (SARS-CoV) emerged in 2002-2003 causing acute respiratory distress syndrome (ARDS) with 10% mortality overall and up to 50% mortality in aged individuals. Middle Eastern Respiratory Syndrome Coronavirus (MERS-CoV) emerged in the Middle East in April of 2012, manifesting as severe pneumonia, acute respiratory distress syndrome (ARDS) and acute renal failure. The virus is still circulating and has been shown to have a mortality rate of ˜49%. Platforms for generating reagents and therapeutics are needed to detect and control the emergence of new strains, especially early in an outbreak prior to the development of type specific serologic reagents and therapeutics.

The present invention overcomes previous shortcomings in the art by providing methods and compositions for detecting coronavirus and identifying it by subgroup and for treating/and or preventing diseases and disorders caused by infection by a coronavirus.

### SUMMARY OF THE INVENTION

The present invention provides a method of detecting the presence of a coronavirus in a sample and identifying the subgroup of the coronavirus in the sample, comprising: a) contacting a sample with a panel of proteins comprising: 1) one or more nucleocapsid proteins from a subgroup 2c coronavirus, 2) one or more nucleocapsid proteins from a subgroup 2b coronavirus, 3) one or more nucleocapsid proteins from a subgroup 2a coronavirus, 4) one or more nucleocapsid proteins from a subgroup 2d coronavirus, 5) one or more nucleocapsid proteins from a subgroup 1a coronavirus, 6) one or more nucleocapsid proteins from a subgroup 1b coronavirus, and 7) any combination of (1) through (6) above, under conditions whereby an antigen/antibody complex can form; and b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b. In some embodiments, the method above can further comprise contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

The present invention also provides a method of identifying a coronavirus spike protein for administration to elicit an immune response to coronavirus in a subject infected by a coronavirus and/or a subject at risk of coronavirus infection and/or to a subject for whom eliciting an immune response to a coronavirus is needed or desired, comprising: a) contacting a sample obtained from a subject infected with a coronavirus with a panel of proteins comprising: 1) one or more spike proteins from a subgroup 2c coronavirus, 2) one or more spike proteins from a subgroup 2b coronavirus, 3) one or more spike proteins from a subgroup 2a coronavirus, 4) one or more spike proteins from a subgroup 2d coronavirus, 5) one or more spike proteins from a subgroup 1a coronavirus, 6) one or more spike proteins from a subgroup 1 b coronavirus, and 7) any combination of (1) through (6) above, under conditions whereby an antigen/antibody complex can form; and b) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired. In some embodiments, this method can comprise contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired. In some embodiments, this method can also comprise the step of administering the coronavirus spike protein identified according to the method to the subject of (a) and/or to a subject at risk of coronavirus infection and/or to a subject infected with a coronavirus and/or to a subject for whom eliciting an immune response to a coronavirus is needed or desired.

In further embodiments, the present invention provides a method of identifying a coronavirus spike protein for administration to elicit an immune response to coronavirus in a subject infected with a coronavirus and/or to a subject at risk of coronavirus infection and/or to a subject for whom eliciting an immune response to coronavirus is needed or desired, comprising: a) contacting a sample obtained from a subject known or suspected to be infected with a coronavirus with a panel of proteins comprising: 1) one or more nucleocapsid proteins from a subgroup 2c coronavirus, 2) one or more nucleocapsid proteins from a subgroup 2b coronavirus, 3) one or more nucleocapsid proteins from a subgroup 2a coronavirus, 4) one or more nucleocapsid proteins from a subgroup 2d coronavirus, 5) one or more nucleocapsid proteins from a subgroup 1a coronavirus, 6) one or more nucleocapsid proteins from a subgroup 1b coronavirus, and 7) any combination of (1) through (6) above, under conditions whereby an antigen/antibody complex can form; b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b; c) contacting the sample from the subject with a panel of proteins comprising: 1) one or more spike proteins from a subgroup 2c coronavirus, 2) one or more spike proteins from a subgroup 2b coronavirus, 3) one or more spike proteins from a subgroup 2a coronavirus, 4) one or more spike proteins from a subgroup 2d coronavirus, 5) one or more spike proteins from a subgroup 1a coronavirus, 6) one or more spike proteins from a subgroup 1b coronavirus, and 7) any combination of (1) through (6) above, under conditions whereby an antigen/antibody complex can form; and d) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to coronavirus is needed or desired.

In some embodiments, this method can further comprise contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

In some embodiments, this method can further comprise contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired.

In some embodiments, this method can further comprise the step of administering the coronavirus spike protein identified according to the methods and/or a coronavirus nucleocapsid protein of the coronavirus subgroup identified according to methods to the subject of (a) and/or to a subject infected with a coronavirus and/or to a subject at risk of coronavirus infection and/or to a subject for whom eliciting an immune response to a coronavirus is needed or desired.

Additionally provided herein is a method of identifying an antibody that neutralizes a coronavirus infecting a subject, comprising: a) isolating a coronavirus from a sample of a subject infected with a coronavirus and/or suspected of being infected with a coronavirus; b) contacting the coronavirus of (a) with a panel of antibodies comprising: 1) an antibody reactive with a spike protein from a subgroup 2c coronavirus, 2) an antibody reactive with a spike protein from a subgroup 2b coronavirus, 3) an antibody reactive with a spike protein from a subgroup 2a coronavirus, 4) an antibody reactive with a spike protein from a subgroup 2d coronavirus, 5) an antibody reactive with a spike protein from a subgroup 1a coronavirus, 6) an antibody reactive with a spike protein from a subgroup 1b coronavirus, and 7) any combination of (1) through (6) above, to form respective coronavirus/antibody compositions, each composition comprising a respective antibody of the panel; c) contacting each of the respective coronavirus/antibody compositions of (b) with cells susceptible to coronavirus infection under conditions whereby coronavirus infection can occur; and d) detecting the presence or absence of infection of the cells, whereby absence of detection of infection of the cells contacted with any of the coronavirus/antibody compositions of (b) identifies the antibody of that coronavirus/antibody composition as an antibody that neutralizes the coronavirus infecting the subject.

In some embodiments, this method can further comprise contacting the coronavirus of (a) with an antibody reactive with a spike protein from a subgroup 3 coronavirus to form a coronavirus/antibody and contacting the coronavirus/antibody composition with cells susceptible to coronavirus infection under conditions whereby coronavirus infection can occur; and detecting the presence or absence of infection of the cells, whereby absence of detection of infection of the cells contacted with the coronavirus/antibody composition identifies the antibody of that coronavirus/antibody composition as an antibody that neutralizes the coronavirus infecting the subject.

In some embodiments, this method can further comprise the step of administering the antibody identified according to the methods to the subject of (a) and/or to a subject infected with a coronavirus and/or to a subject at risk of coronavirus infection and/or to a subject for whom eliciting an immune response to a coronavirus is needed or desired.

The foregoing and other objects and aspects of the present invention are explained in detail in the specification set forth below.

### BRIEF DESCRIPTION OF THE DRAWINGS

FIG. 1. Characterization of MERS Eng 1. Growth of MERS-CoV Hu isolates at indicated multiplicities of infection (MOIs) in (A) Vero Cells, (B) Calu-3 Cells. Infected cultures were sampled in triplicates at times indicated, and viral titers (shown as pfu/mL) were determined by plaque assay on Vero cells. Error bars indicate SEM. (C) Northern blot analysis of RNA harvested 12 HPI from Vero cells infected with MERS-CoV Hu isolates at an MOI of 5. (D) Western blots of lysates harvested 12 HPI from Calu-3 2B4 cells infected with MERS-CoV Hu isolates at an MOI of 5, were probed with antisera to S and N proteins. Various forms of S proteins are indicated. β-actin indicates loading control.

FIG. 2. S vaccine, but not N vaccine generates neutralizing antibodies to MERS-CoV Hu isolates that are out competed NA01 patient sera. (A) Serum from each of 4 young and aged mice immunized with VRP S or VRP N were tested in a PRNT₅₀ assay to neutralize MERS-CoV Hu isolates. Error bars indicate SEM. (B and C) NA01 patient sera collected at indicated dates post hospitalization were analyzed in an ELISA assay using cell lysates expressing S and N antigens from VRPs. (D) Indicated dilutions of NA01 patient sera were screened with 1:800 dilutions of mouse antisera to S, N, BtCoV HKU5.5 N, or SARS-CoV S in an in vitro competition assay for binding to MERS-CoV or SARS-CoV, (E) Phylogenetic tree generated using full-length genome sequences of 51 CoVs shows that CoVs are divided into three distinct phylogenetic groups defined as α, β, and γ. This taxonomical nomenclature replaced the former group 1, 2 and 3 designation, respectively. Classical subgroup clusters were marked as 21-2d for the β-CoVs and 1a and 1b for the α-CoVs. The tree was generated using Maximum Likelihood with the PhyML package. The scale bar represents nucleotide substitutions. Only nodes with bootstrap support above 70% were labeled.

FIG. 3. Cross reactivity and Cross Neutralization of Mouse Antisera to N and S Proteins of Group 2c bat viruses with MERS-CoV Hu isolates. Western blots showing cross reactivity between N (A and B) and S (C and D) proteins of MERS-CoVHu isolates and N and S proteins of BtCoV HKU4.2, and HKU 5.5 (E). PRNT₅₀ assays showing absence of cross neutralization of MERS-CoVHu isolates by antisera to BtCoVHKU4.2 and 5.5 S proteins. Serum from groups of 4 mice immunized with VRPs was tested in this assay. Error bars indicate SEM. Note the cross reactivity of antisera to BtCoV HKU5.5 S protein to S proteins of MERS-CoV Hu isolates (D), but no cross neutralization.

FIG. 4. Cross reactivity and Cross Neutralization of Mouse Antisera to N and S Proteins of SARS-CoV viruses with MERS-CoV Hu isolates. Western blots showing no cross reactivity between N (A) and S (B) proteins of MERS-CoVHu isolates and SARS-CoV. (C). PRNT₅₀ assays showing absence of cross neutralization of MERS-CoV Hu isolates by antisera to SARS-CoV S, and SARS-CoV by antisera to MERS-CoV/SA-1/2012 S protein and BtCoV 279 S protein. Note that antisera to SARS-CoV S neutralize SARS-CoV. Serum from groups of 4 mice immunized with VRPs was tested in this assay and error bars indicate SEM. (D) ELISA results showing the absence of reactivity of NA01 patient sera to SARS-CoV S antigen.

FIG. 5. Cross reactivity and Cross Neutralization of Mouse Antisera to N and S Proteins of Group 2b Bat viruses with MERS-CoV Hu isolates. Western blots showing cross reactivity between N (A and B) and S (C and D) proteins of MERS-CoVHu isolates and N and S proteins of BtCoV 279 and HKU 3. (E) PRNT₅₀ assays showing absence of cross neutralization of MERS-CoVHu isolates by antisera to BtCoV 279S and HKU 3 S proteins. Serum from groups of 4 mice immunized with VRPs was tested in this assay. Error bars indicate SEM.

FIG. 6. Serology with Human and Mouse sera to other respiratory viruses shows group specific cross reactivity. (A) NA01 patient sera collected at indicated dates were analyzed in an ELISA assay using cell lysates expressing indicated antigens. (B) Mouse antisera to indicated antigens were screened in an ELISA assay HCoVs NL63, OC43 and SARS-CoV. (C) Human antisera to indicated CoVs were screened in an ELISA with cell lysates expressing indicated antigens.

FIG. 7. Cross reactivity and Cross Neutralization of Mouse Antisera to S Proteins of alpha CoVs and MERS CoVHu isolates. Western blots showing cross reactivity between S proteins of (A) BtCoV HKU 2.298 and (B) BtCoV 1A MERS-CoVHu isolates. (C) PRNT₅₀ assays showing absence of cross neutralization of MERS-CoVHu isolates by antisera. Serum from groups of four mice immunized with VRPs was tested in this assay. Error bars indicate SEM.

FIG. 8. Expression of MERS-COV Hu/SA-N1/2012 S and N proteins from VRPs. (A) Lysates from Vero Cells infected with VRPs expressing S and N proteins of MERS SA 1, or MERS-CoV isolates were probed on a Western blot using antisera against S and N proteins of MERS SA 1.

FIG. 9. Schematic of the genome organization of MERS SA 1, with indicated open reading frames (ORFs). Mutations in MERS Eng 1 in the respective ORFs are indicated in red colored ball sticks. Detailed description of the mutation at the nucleotide and amino acid levels are indicated in the table.

### DETAILED DESCRIPTION OF THE INVENTION

The present invention is based on the unexpected discovery that the subgroup of a coronavirus can be identified using a method of this invention, which allows, for example, for rapid and early detection of an emerging coronavirus. Such detection and identification of the coronavirus by subgroup allows for rapid response and treatment/prophylaxis by employing this information in the selection of a vaccine and/or therapeutic for administration not only to an infected subject, but to others at risk of infection and/or in need of treatment of or protection from coronavirus infection as soon as the subgroup of the coronavirus is known. For example, when an emerging coronavirus is detected in one or more subjects of a population, a rapid determination of the subgroup of the emerging coronavirus can be made according to the methods of this invention and an appropriate therapeutic and/or immunogen can be administered to infected subjects, as well as subjects (e.g., in the same community or environment or population with infected subjects) at risk of infection and/or other subjects who need or desire such a therapeutic or immunogen, during in the early phase of detection of the emerging coronavirus, thereby minimizing the potential for and possibility of epidemic or pandemic coronavirus infection. The broadly applicable platform approach described herein provides reagents, antisera, and vaccines (immunogens) that allow for rapid diagnosis and intervention against emerging coronaviruses, such as MERS-CoV and other zoonotic coronaviruses of the future.

The present invention provides a method of detecting the presence of a coronavirus in a sample and identifying the subgroup of the coronavirus in the sample, comprising: a) contacting a sample with a panel of proteins comprising: 1) one or more nucleocapsid proteins from a subgroup 2c coronavirus, 2) one or more nucleocapsid proteins from a subgroup 2b coronavirus, 3) one or more nucleocapsid proteins from a subgroup 2a coronavirus, 4) one or more nucleocapsid proteins from a subgroup 2d coronavirus, 5) one or more nucleocapsid proteins from a subgroup 1a coronavirus, 6) one or more nucleocapsid proteins from a subgroup 1b coronavirus, and 7) any combination of (1) through (6) above, under conditions whereby an antigen/antibody complex can form; and b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b. In some embodiments, the method above can further comprise contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

The present invention also provides a method of identifying a coronavirus spike protein for administration to elicit an immune response to coronavirus in a subject infected by a coronavirus and/or a subject at risk of coronavirus infection and/or to a subject for whom eliciting an immune response to a coronavirus is needed or desired, comprising: a) contacting a sample obtained from a subject infected with a coronavirus with a panel of proteins comprising: 1) one or more spike proteins from a subgroup 2c coronavirus, 2) one or more spike proteins from a subgroup 2b coronavirus, 3) one or more spike proteins from a subgroup 2a coronavirus, 4) one or more spike proteins from a subgroup 2d coronavirus, 5) one or more spike proteins from a subgroup 1a coronavirus, 6) one or more spike proteins from a subgroup 1b coronavirus, and 7) any combination of (1) through (6) above, under conditions whereby an antigen/antibody complex can form; and b) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired. In some embodiments, this method can comprise contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired. In some embodiments, this method can also comprise the step of administering the coronavirus spike protein identified according to the method to the subject of (a) and/or to a subject at risk of coronavirus infection and/or to a subject infected with a coronavirus and/or to a subject for whom eliciting an immune response to a coronavirus is needed or desired.

In further embodiments, the present invention provides a method of identifying a coronavirus spike protein for administration to elicit an immune response to coronavirus in a subject infected with a coronavirus and/or to a subject at risk of coronavirus infection and/or to a subject for whom eliciting an immune response to coronavirus is needed or desired, comprising: a) contacting a sample obtained from a subject known or suspected to be infected with a coronavirus with a panel of proteins comprising: 1) one or more nucleocapsid proteins from a subgroup 2c coronavirus, 2) one or more nucleocapsid proteins from a subgroup 2b coronavirus, 3) one or more nucleocapsid proteins from a subgroup 2a coronavirus, 4) one or more nucleocapsid proteins from a subgroup 2d coronavirus, 5) one or more nucleocapsid proteins from a subgroup 1a coronavirus, 6) one or more nucleocapsid proteins from a subgroup 1b coronavirus, and 7) any combination of (1) through (6) above, under conditions whereby an antigen/antibody complex can form; b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b; c) contacting the sample from the subject with a panel of proteins comprising: 1) one or more spike proteins from a subgroup 2c coronavirus, 2) one or more spike proteins from a subgroup 2b coronavirus, 3) one or more spike proteins from a subgroup 2a coronavirus, 4) one or more spike proteins from a subgroup 2d coronavirus, 5) one or more spike proteins from a subgroup 1a coronavirus, 6) one or more spike proteins from a subgroup 1b coronavirus, and 7) any combination of (1) through (6) above, under conditions whereby an antigen/antibody complex can form; and d) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to coronavirus is needed or desired.

In some embodiments, this method can further comprise contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

In some embodiments, this method can further comprise contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired.

In some embodiments, this method can further comprise the step of administering the coronavirus spike protein identified according to the methods and/or a coronavirus nucleocapsid protein of the coronavirus subgroup identified according to methods to the subject of (a) and/or to a subject infected with a coronavirus and/or to a subject at risk of coronavirus infection and/or to a subject for whom eliciting an immune response to a coronavirus is needed or desired.

Additionally provided herein is a method of identifying an antibody that neutralizes a coronavirus infecting a subject, comprising: a) isolating a coronavirus from a sample of a subject infected with a coronavirus and/or suspected of being infected with a coronavirus; b) contacting the coronavirus of (a) with a panel of antibodies comprising: 1) an antibody reactive with a spike protein from a subgroup 2c coronavirus, 2) an antibody reactive with a spike protein from a subgroup 2b coronavirus, 3) an antibody reactive with a spike protein from a subgroup 2a coronavirus, 4) an antibody reactive with a spike protein from a subgroup 2d coronavirus, 5) an antibody reactive with a spike protein from a subgroup a coronavirus, 6) an antibody reactive with a spike protein from a subgroup 1b coronavirus, and 7) any combination of (1) through (6) above, to form respective coronavirus/antibody compositions, each composition comprising a respective antibody of the panel; c) contacting each of the respective coronavirus/antibody compositions of (b) with cells susceptible to coronavirus infection under conditions whereby coronavirus infection can occur; and d) detecting the presence or absence of infection of the cells, whereby absence of detection of infection of the cells contacted with any of the coronavirus/antibody compositions of (b) identifies the antibody of that coronavirus/antibody composition as an antibody that neutralizes the coronavirus infecting the subject.

In some embodiments, this method can further comprise contacting the coronavirus of (a) with an antibody reactive with a spike protein from a subgroup 3 coronavirus to form a coronavirus/antibody and contacting the coronavirus/antibody composition with cells susceptible to coronavirus infection under conditions whereby coronavirus infection can occur; and detecting the presence or absence of infection of the cells, whereby absence of detection of infection of the cells contacted with the coronavirus/antibody composition identifies the antibody of that coronavirus/antibody composition as an antibody that neutralizes the coronavirus infecting the subject.

In some embodiments, this method can further comprise the step of administering the antibody identified according to the methods to the subject of (a) and/or to a subject infected with a coronavirus and/or to a subject at risk of coronavirus infection and/or to a subject for whom eliciting an immune response to a coronavirus is needed or desired.

Nonlimiting examples of a subgroup 1a coronavirus of this invention include FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_001961.1; GenBank Accession No. DQ811787), as well as any other subgroup 1a coronavirus now known (e.g., as can be found in the GenBank® Database) or later identified, and any combination thereof.

Nonlimiting examples of a subgroup 1b coronavirus of this invention include BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. JN601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank Database) or later identified, and any combination thereof.

Nonlimiting examples of a subgroup 2a coronavirus of this invention include HCoV.HKU1.C.N5 (GenBank Accession No. DQ339101), MHV.A59 (GenBank Accession No. NC_001846), PHEV.VW572 (GenBank Accession No. NC_007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

Nonlimiting examples of a subgroup 2b coronavirus of this invention include BtSARS.HKU3.1 (GenBank Accession No, DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GD01 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), SARSCoV.MA.15 (GenBank Accession No. DQ497008), as well as any other subgroup 2b coronavirus now known (e.g., as can be found in the GenBank® Database) or later identified, and any combination thereof.

Nonlimiting examples of a subgroup 2c coronavirus of this invention include Middle East respiratory syndrome coronavirus isolate Riyadh 22012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa_152013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa 19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_122013 (GenBank Accession No. KF600627.1), Middle East respiratory syndrome coronavirus isolate Bisha≦1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA 003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA 002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV133.2005 (GenBank Accession No. NC_008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012, (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No: KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No: KC522046, 1, GenBank Accession No: KC522045.1, GenBank Accession No: KC522044.1, GenBank Accession No: KC522043.1, GenBank Accession No: KC522042.1, GenBank Accession No: KC522041.1, GenBank Accession No: KC522040.1 GenBank Accession No: KC522039.1, GenBank Accession No: KC522038.1, GenBank Accession No: KC522037.1, GenBank Accession No: KC522036.1, GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040, 1, GenBank Accession No: KC522039.1 GenBank Accession No: KC522038.1 GenBank Accession No: KC522037.1 GenBank Accession No: KC522036.1, GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1, GenBank Accession No: KC522063.1, GenBank Accession No: KC522062.1), as well as any other subgroup 2c coronavirus now known (e.g., as can be found in the GenBank® Database) or later identified, and any combination thereof.

Nonlimiting examples of a subgroup 2d coronavirus of this invention include BtCoV.HKU9.2 (GenBank Accession No. EF065514), BtCoV.HKU9.1 (GenBank Accession No. NC_009021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

Nonlimiting examples of a subgroup 3 coronavirus of this invention include IBV.Beaudette.IBV.p65 (GenBank Accession No. DQ001339), as well as any other subgroup 3 coronavirus now known (e.g., as can be found in the GenBank® Database) or later identified, and any combination thereof.

The coronaviruses in the respective subgroups 1a, 1b, 2a, 2b, 2c, 2d and 3 can be included in the methods and compositions of this invention in any combination, as would be well understood to one of ordinary skill in the art.

As used herein, “a” or “an” or “the” can mean one or more than one. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP §2111.03.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

In the methods of this invention in which formation of an antigen/antibody complex is detected, a variety of assays can be employed for such detection. For example, various immunoassays can be used to detect antibodies or proteins (antigens) of this invention. Such immunoassays typically involve the measurement of antigen/antibody complex formation between a protein or peptide (i.e., an antigen) and its specific antibody.

The immunoassays of the invention can be either competitive or noncompetitive and both types of assays are well-known and well-developed in the art. In competitive binding assays, antigen or antibody competes with a detectably labeled antigen or antibody for specific binding to a capture site bound to a solid surface. The concentration of labeled antigen or antibody bound to the capture agent is inversely proportional to the amount of free antigen or antibody present in the sample.

Noncompetitive assays of this invention can be, for example, sandwich assays, in which, for example, the antigen is bound between two antibodies. One of the antibodies is used as a capture agent and is bound to a solid surface. The other antibody is labeled and is used to measure or detect the resultant antigen/antibody complex by e.g., visual or instrument means. A number of combinations of antibody and labeled antibody can be used, as are well known in the art. In some embodiments, the antigen/antibody complex can be detected by other proteins capable of specifically binding human immunoglobulin constant regions, such as protein A, protein L or protein G. These proteins are normal constituents of the cell walls of streptococcal bacteria. They exhibit a strong nonimmunogenic reactivity with immunoglobulin constant regions from a variety of species. (See, e.g., Kronval et al. J. Immunol. 111:1401-1406 (1973); Akerstrom et al. J. Immunol. 135:2589-2542 (1985)).

In some embodiments, the non-competitive assays need not be sandwich assays. For instance, the antibodies or antigens in the sample can be bound directly to the solid surface. The presence of antibodies or antigens in the sample can then be detected using labeled antigen or antibody, respectively.

In some embodiments, antibodies and/or proteins can be conjugated or otherwise linked or connected (e.g., covalently or noncovalently) to a solid support (e.g., bead, plate, slide, dish, membrane or well) in accordance with known techniques. Antibodies can also be conjugated or otherwise linked or connected to detectable groups such as radiolabels (e.g., ³⁵S, ¹²⁵I, ³²P, ¹³H, ¹⁴C, ¹³¹I), enzyme labels (e.g., horseradish peroxidase, alkaline phosphatase), gold beads, chemiluminescence labels, ligands (e.g., biotin) and/or fluorescence labels (e.g., fluorescein) in accordance with known techniques.

A variety of organic and inorganic polymers, both natural and synthetic can be used as the material for the solid surface. Nonlimiting examples of polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that can be used include, but are not limited to, paper, glass, ceramic, metal, metalloids, semiconductive materials, cements and the like. In addition, substances that form gels, such as proteins (e.g., gelatins), lipopolysaccharides, silicates, agarose and polyacrylamides can be used. Polymers that form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes can be employed depending upon the nature of the system.

A variety of immunoassay systems can be used, including but not limited to, radio-immunoassays (RIA), enzyme-linked immunosorbent assays (ELISA) assays, enzyme immunoassays (EIA), “sandwich” assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, immunoprecipitation, competitive binding assays and immunohistochemical staining. These and other assays are described, among other places, in Hampton et al. (Serological Methods, a Laboratory Manual, APS Press, St Paul, Minn. (1990)) and Maddox et al. (J. Exp. Med. 158:1211-1216 (1993); the entire contents of which are incorporated herein by reference for teachings directed to immunoassays).

The methods of this invention can also be carried out using a variety of solid phase systems, such as described in U.S. Pat. No. 5,879,881, as well as in a dry strip lateral flow system. (e.g., a “dipstick” system), such as described, for example, in U.S. Patent Publication No. 20030073147, the entire contents of each of which are incorporated by reference herein.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody or bispecific antibody.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

Monoclonal antibodies can be produced in a hybridoma cell line according to the technique of Kohler and Milstein, (1975) Nature 265:495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacterial cell such as E. coli by recombinant techniques known to those skilled in the art. See, e.g., W. Huse, (1989) Science 246:1275-81.

Antibodies can also be obtained by phage display techniques known in the art or by immunizing a heterologous host with a cell containing an epitope of interest.

“Nidovirus” as used herein refers to viruses within the order Nidovirales, including the families Coronaviridae and Arteriviridae. All viruses within the order Nidovirales share the unique feature of synthesizing a nested set of multiple subgenomic mRNAs. See M. Lai and K. Holmes, Coronaviridae: The Viruses and Their Replication, in Fields Virology, pg 1163, (4^{th} Ed. 2001). Particular examples of Coronaviridae include, but are not limited to, toroviruses and coronaviruses.

“Coronavirus” as used herein refers to a genus in the family Coronaviridae, which family is in turn classified within the order Nidovirales. The coronaviruses are large, enveloped, positive-stranded RNA viruses. They have the largest genomes of all RNA viruses and replicate by a unique mechanism that results in a high frequency of recombination. The coronaviruses include antigenic groups I, II, and III. Nonlimiting examples of coronaviruses include SARS coronavirus, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras of any of the foregoing. See Lai and Holmes “Coronaviridae: The Viruses and Their Replication” in Fields Virology, (4^{th} Ed. 2001).

A “nidovirus permissive cell” or “coronavirus permissive cell” as used herein can be any cell in which a coronavirus can at least replicate, including both naturally occurring and recombinant cells. In some embodiments the permissive cell is also one that the nidovirus or coronavirus can infect. The permissive cell can be one that has been modified by recombinant means to produce a cell surface receptor for the nidovirus or coronavirus.

A “heterologous RNA” as described herein can encode any protein, peptide, antisense sequence, ribozyme, etc., to be administered to a subject of this invention for any purpose. For example, the heterologous RNA can encode, and be expressed in the subject to produce, a protein or peptide. The protein or peptide may, for example, be an antigen or immunogen in embodiments where it is desired to produce antibodies in an animal subject, which antibodies can be collected and used for diagnostic and/or therapeutic purposes, or where it is desired to elicit an immune response to the protein or peptide in a subject.

A “structural protein” as used herein refers to a protein required for production of coronavirus particles of this invention, such as those encoded by the S, E, M and N genes, as well as any other structural proteins now known or later identified in the coronavirus and in particular in the SARS virus genome. In embodiments of this invention wherein the replicon RNA and/or helper RNAs lack a nucleotide sequence encoding a structural protein, the nucleotide sequence can be wholly or partly deleted, or the sequence can be present but in a mutated form, so that the net effect is that the replicon RNA and/or the helper RNA is effectively incapable of producing the necessary structural protein in functional form. Thus, for example, in an embodiment that recites a replicon RNA or helper RNA that “lacks a sequence encoding at least one coronavirus structural protein,” it is meant that the nucleotide sequence encoding the at least one coronavirus structural protein is deleted completely or in part from the replicon RNA or helper RNA or it is meant that the nucleotide sequence encoding the at least one coronavirus structural protein is present on the replicon RNA or helper RNA but in a form (e.g., mutated or otherwise altered) that cannot be expressed to produce a functional protein.

“Multiplication-defective” or “replication-defective” as used herein means that the replicon RNA contained within viral particles produced according to the present invention does not itself contain sufficient genetic information to allow for the production of new infectious viral particles.

An “isolated” nucleic acid molecule is one that is chemically synthesized (e.g., derived from reverse transcription) or is separated from other nucleic acid molecules that are present in the natural source of the nucleic acid molecule. Preferably, an “isolated” nucleic acid molecule is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. (e.g., as described in Sambrook et al., eds., “Molecular Cloning: A Laboratory Manual,” 2nd ed., Cold Spring Harbor Laboratory, Cold Spring Harbor Laboratory Press, Cold Spring Harbor, N.Y., 1989).

In particular embodiments, a nucleic acid of this invention has at least about 50%, 60%, 70%, 75%, 80%, 85%, 90%, 95%, 98% or more nucleic acid sequence homology with the sequences specifically disclosed herein. The term “homology” as used herein refers to a degree of similarity between two or more sequences. There can be partial homology or complete homology (i.e., identity). A partially homologous sequence that at least partially inhibits an identical sequence from hybridizing to a target nucleic acid is referred to using the functional term “substantially homologous.” The inhibition of hybridization to the target sequence can be examined using a hybridization assay (Southern or northern blot, solution hybridization and the like) under conditions of low stringency. A substantially homologous sequence or hybridization probe will compete for and inhibit the binding of a completely homologous sequence to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding can be tested by the use of a second target sequence, which lacks even a partial degree of complementarity (e.g., less than about 30% identity). In the absence of non-specific binding, the probe will not hybridize to the second non-complementary target sequence.

Alternatively stated, in particular embodiments, nucleic acids encoding a cDNA of a coronavirus that hybridize under the conditions described herein to the complement of the sequences specifically disclosed herein can also be used according to the present invention. The term “hybridization” as used herein refers to any process by which a first strand of nucleic acid binds with a second strand of nucleic acid through base pairing.

The term “stringent” as used here refers to hybridization conditions that are commonly understood in the art to define the commodities of the hybridization procedure. High stringency hybridization conditions that will permit homologous nucleotide sequences to hybridize to a nucleotide sequence as given herein are well known in the art. As one example, hybridization of such sequences to the nucleic acid molecules disclosed herein can be carried out in 25% formamide, 5×SSC, 5×Denhardt's solution and 5% dextran sulfate at 42° C., with wash conditions of 25% formamide, 5×SSC and 0.1% SDS at 42° C., to allow hybridization of sequences of about 60% homology. Another example includes hybridization conditions of 6×SSC, 0.1% SDS at about 45° C., followed by wash conditions of 0.2×SSC, 0.1% SDS at 50-65° C. Another example of stringent conditions is represented by a wash stringency of 0.3 M NaCl, 0.03M sodium citrate, 0.1% SDS at 60-70° C. using a standard hybridization assay (see SAMBROOK et al., EDS., MOLECULAR CLONING: A LABORATORY MANUAL 2d ed. (Cold Spring Harbor, N.Y. 1989, the entire contents of which are incorporated by reference herein).

Also provided herein are an RNA molecule and a coronavirus particle produced by a cDNA of this invention and a coronavirus particle comprising the RNA produced from the cDNA of this invention. Further provided herein is a vector comprising the cDNA or RNA of this invention and a cell comprising the vector of this invention.

In further embodiments, the present invention provides a method of eliciting an immune response in a subject, comprising administering to, delivering to, and/or introducing into the subject an effective amount of the nucleic acids, viruses, particles, compositions, antibodies and/or populations of this invention.

Also provided herein is a method of treating and/or preventing a nidovirus (e.g., a coronavirus) infection and/or a disorder or diseases caused by infection by a nidovirus (e.g., a coronavirus) in a subject, comprising administering, delivering and/or introducing into the subject an effective amount of a nucleic acid, virus, particle, protein, peptide, immunogen, composition, antibody and/or population of this invention.

The nucleic acids, proteins, peptides, viruses, vectors, particles, antibodies and populations of this invention are intended for use as therapeutic agents and immunological reagents, for example, as antigens, immunogens, vaccines, and/or nucleic acid delivery vehicles. Thus, in various embodiments, the present invention provides a composition comprising the nucleic acid, virus, vector, particle, antibody and/or population of this invention in a pharmaceutically acceptable carrier. The compositions described herein can be formulated for use as reagents (e.g., to produce antibodies) and/or for administration in a pharmaceutical carrier in accordance with known techniques. See, e.g., Remington, The Science And Practice of Pharmacy (latest edition).

In embodiments of this invention wherein a coronavirus protein is being administered, delivered and/or introduced into a subject, e.g., to elicit or induce an immune response, the protein can be administered, delivered and/or introduced into the subject as a protein present in an inactivated (e.g., inactivated through UV irradiation or formalin treatment) coronavirus. The protein or active fragment thereof of this invention can be administered, delivered and/or introduced into the subject according to any method now known or later identified for administration, introduction and/or delivery of protein or active fragment thereof, as would be well known to one of ordinary skill in the art. Nonlimiting examples include administration of the protein or fragment with a protease inhibitor or other agent to protect it from degradation and/or with a polyalkylene glycol moiety (e.g., polyethylene glycol).

In some embodiments, the coronavirus protein or active fragment thereof can be administered to a subject as a nucleic acid molecule, which can be a naked nucleic acid molecule or a nucleic acid molecule present in a vector (e.g., a delivery vector, which in some embodiments can be a VRP). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral and fungal vectors, liposomal delivery agents, nanoparticles, and gene gun related-mechanisms.

As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

As one example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 10¹², 10¹⁵ and/or 10²⁰ pfu per injection. Ideally, a subject will receive a single injection. If additional injections are necessary, they can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired immunological response.

The exact amount of the nucleic acid or vector required will vary from subject to subject, depending on the species, age, weight and general condition of the subject, the particular nucleic acid or vector used, its mode of administration and the like. Thus, it is not possible to specify an exact amount for every nucleic acid or vector. However, an appropriate amount can be determined by one of ordinary skill in the art using only routine experimentation given the teachings herein.

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

Parenteral administration of the peptides, polypeptides, nucleic acids and/or vectors of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained. See, e.g., U.S. Pat. No. 3,610,795, which is incorporated by reference herein in its entirety.

In the manufacture of a pharmaceutical composition according to embodiments of the present invention, the composition of this invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents.

The carrier may be a solid or a liquid, or both, and is preferably formulated with the composition of this invention as a unit-dose formulation. The pharmaceutical compositions are prepared by any of the well-known techniques of pharmacy including, but not limited to, admixing the components, optionally including one or more accessory ingredients. Exemplary pharmaceutically acceptable carriers include, but are not limited to, sterile pyrogen-free water and sterile pyrogen-free physiological saline solution. Such carriers can further include protein (e.g., serum albumin) and sugar (sucrose, sorbitol, glucose, etc.)

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration. The compositions herein may also be administered via a skin scarification method, or transdermally via a patch or liquid. The compositions may be delivered subdermally in the form of a biodegradable material that releases the compositions over a period of time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

A subject of this invention is any animal that is susceptible to infection by coronavirus and/or susceptible to diseases or disorders caused by coronavirus infection. A subject of this invention can be a mammal and in particular embodiments is a human, which can be an infant, a child, an adult or an elderly adult. A “subject at risk of infection by a coronavirus” or a “subject at risk of coronavirus infection” is any subject who may be or has been exposed to a coronavirus.

As used herein, an “effective amount” refers to an amount of a compound or composition that is sufficient to produce a desired effect, which can be a therapeutic, prophylactic and/or beneficial effect.

Thus, the present invention provides a method of inducing or eliciting an immune response in a subject, comprising administering to the subject an effective amount of a virus, vector, particle, population and/or composition of this invention.

The present invention also provides a method of treating and/or preventing a coronavirus infection in a subject, comprising administering to the subject an effective amount of a virus, vector, particle, population and/or composition of this invention.

Also as used herein, the terms “treat,” “treating” and “treatment” include any type of mechanism, action or activity that results in a change in the medical status of a subject, including an improvement in the condition of the subject (e.g., change or improvement in one or more symptoms and/or clinical parameters), delay in the progression of the condition, delay of the onset of a disease or illness, etc.

One nonlimiting example of an effective amount of a virus or virus particle (e.g., VRP) of this invention is from about 10⁴ to about 10¹⁰, preferably from about 10⁵ to about 10⁹, and in particular from about 10⁶ to about 10⁸ infectious units (IU, as measured by indirect immunofluorescence assay), or virus particles, per dose, which can be administered to a subject, depending upon the age, species and/or condition of the subject being treated. For subunit vaccines (e.g., purified antigens) a dose range of from about 1 to about 100 micrograms can be used. As would be well known to one of ordinary skill in the art, the optimal dosage would need to be determined for any given antigen or vaccine, e.g., according to the method of production and resulting immune response.

For administration of serum or antibodies, as one nonlimiting example, a dosage range of from about 20 to about 40 international Units/Kilogram can be used, although it would be well understood that optimal dosage for administration to a subject of this invention needs to be determined, e.g., according to the method of production and resulting immune response.

In some embodiments of the present invention, the compositions can be administered with an adjuvant. As used herein, “adjuvant” describes a substance, which can be any immunomodulating substance capable of being combined with the polypeptide or nucleic acid vaccine to enhance, improve or otherwise modulate an immune response in a subject without deleterious effect on the subject.

Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN™. adjuvant, saponin, Quil A or other saponin fraction, monophosphoryl lipid A, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN™ 85 detergent (ICI Surfactants), 0.7% (v/v) TWEEN™ 80 detergent (ICI Surfactants), 2.5% (v/v) ethanol, 200 pg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN™ 85 detergent, 0.7% (v/v) Tween 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines.

In some embodiments, VEE replicon vectors can be used to express coronavirus structural genes in producing combination vaccines. Dendritic cells, which are professional antigen-presenting cells and potent inducers of T-cell responses to viral antigens, are preferred targets of VEE and VEE replicon particle infection, while SARS coronavirus targets the mucosal surfaces of the respiratory and gastrointestinal tract. As the VEE and coronavirus replicon RNAs synergistically interact, two-vector vaccine systems are feasible that may result in increased immunogenicity when compared with either vector alone. Combination prime-boost vaccines (e.g., DNA immunization and vaccinia virus vectors) have dramatically enhanced the immune response (notably cellular responses) against target papillomavirus and lentivirus antigens compared to single-immunization regimens (Chen et al. (2000) Vaccine 18:2015-2022; Gonzalo et al. (1999) Vaccine 17:887-892; Hanke et al. (1998) Vaccine 16:439-445; Pancholi et al. (2000) J Infect. Dis. 182:18-27). Using different recombinant viral vectors (influenza and vaccinia) to prime and boost may also synergistically enhance the immune response, sometimes by an order of magnitude or more (Gonzalo, et al. (1999) Vaccine 17:887-892). Thus, the present invention also provides methods of combining different recombinant viral vectors (e.g., VEE and coronavirus) in prime boost protocols.

The present invention further provides a kit for carrying out the methods of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other solutions and instructions for using the kit, as would be well known in the art. Such kits can further comprise pharmaceutical compositions, adjuvants and/or other immunostimulatory or immunomodulating agents, as are well known in the art.

Although the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

When the components of the kit are provided in one or more liquid solutions, the liquid solution can be an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container. For example, wherein the components of the kit are in lyophilized form, the kit may optionally contain a sterile and physiologically acceptable reconstitution medium such as water, saline, buffered saline, and the like.

In some embodiments, the containers of the kit can include at least one vial, test tube, flask, bottle, syringe, finger prick test or other containers, into which the reagents, antibodies, antigens, and/or compositions/formulations of the present invention, and any other desired agent, may be placed and suitably aliquoted. Where separate components are included, the kit will also generally contain at least a second container into which these are placed, enabling the administration of separated designed doses or amounts. The kits may also comprise additional containers for containing a sterile, pharmaceutically acceptable buffer or other diluent.

The terms “kit” and “system,” as used herein refer, e.g., to combinations of detection reagents, or one or more detection reagents in combination with one or more other types of elements or components (e.g., other types of biochemical reagents, containers, packages such as packaging intended for commercial sale, substrates to which detection reagents are attached, electronic hardware components, etc.)

Another form of kit included in the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one detection reagent for detecting one or more antibodies or antigens or other proteins of the present invention, one or more containers which contain wash reagents (such as phosphate buffered saline, Tris-buffers, etc.), and one or more containers which contain the reagents used to reveal the presence of the bound antibody or antigen and/or other detection reagents.

A kit of this invention can further comprise therapeutic agents and/or compositions that can be used, for example in a treatment protocol and/or prophylactic treatment protocol for a subject identified according to the methods described herein as a subject in need thereof. As used herein, a “prophylactic treatment” describes the use of medication and/or other intervention and/or other therapy before the clinical manifestation of the disease or disorder.

The present invention also provides a computer program product comprising: a computer readable storage medium having computer readable code embodied in the medium, the computer code comprising: computer readable code to perform operations to carry out the methods of this invention.

Further provided herein is a computer system, comprising: a processor; and a memory coupled to the processor, the memory comprising computer readable program code embodied therein that, when executed by the processor, causes the processor to perform operations to carry out the methods of this invention.

As noted above, a kit of this invention can comprise electronic hardware components. In some embodiments of this invention, the electronic hardware may perform and/or support functionality that corresponding to various operations described herein. For example, functions described and/or illustrated in diagrams and/or flowchart illustrations of methods, apparatus (systems) and/or computer program products according to some embodiments may be performed by the electronic hardware. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, infrared, or semiconductor system, apparatus, or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

The examples, which follow, are set forth to illustrate the present invention, and are not to be construed as limiting thereof. In the following examples, mM means milli molar, pg means microgram, ml means milliliter, μl means microliter, V means volt, μF means microfarad, cm means centimeter, h means hour, ORF means open reading frame, GFP means green fluorescent protein, PBS means phosphate-buffered saline, M means molar, s means second, nt means nucleotide, and min means minute.

### EXAMPLES

### Example 1

### Platform Strategies for Rapid Response Against Emerging Coronaviruses: MERS CoV Serologic and Antigenic Relationships in Vaccine Design

Phylogenetic analysis groups CoVs into four genera; Alpha, Beta, Gamma and Delta, and for most mammalian coronaviruses, bats are considered reservoirs. SARS-CoV is closely related to BtCoV HKU3 while MERS-CoV is closely related to Pipistrellus BtCoV HKU5 and Tylonycteris BtCoV HKU4. However, the serologic and antigenic relationship between strains is unclear. Given the vast number of genetically distinct CoVs, well defined serologic and virological reagents are needed to rapidly track MERS-CoV and other coronavirus infections in natural populations and to optimize vaccine and therapeutic designs early in an outbreak setting, especially within and between phylogenetic clusters. The spike (S) and the nucleocapsid (N) proteins are the major immunogenic components of CoVs and are produced in abundant quantities during infection. Therefore, antibodies against S and N protein have sensitive diagnostic and therapeutic potential.

In the studies described herein, alphavirus replicon vaccine vectors are used to express a panel of recombinant S glycoprotein and N proteins from distantly related alpha and beta coronaviruses, including MERS-CoV and other closely related group 2c coronaviruses. Using mouse polyclonal antisera and recombinant proteins, the cross reactivity and neutralization titers of these antisera are compared between distant human, animal and Bat CoVs. These results indicate that i) the spike (S) glycoprotein, but not the nucleocapsid (N) protein is the major determinant of neutralizing antibody response to MERS-CoV; ii) the N proteins of coronaviruses (CoVs) only cross react within, but not between subgroups; iii) little if any cross neutralization or cross reactivity exists between the S proteins of CoVs within Group 2c or any other subgroup; and iv) cross neutralization and cross-reactive patterns were validated with the convalescent serum from MERS-CoV infected NA01 patient (origin of MERS-CoV Hu/England-N1/2012), and a donor panel of human serum against three different human coronaviruses.

Middle East Respiratory Syndrome Coronavirus (MERS-CoV) emerged in 2012 causing Severe Acute Respiratory Disease and pneumonia, with ˜44% mortality in 136 cases identified to date. Design of vaccines to limit the virus spread or diagnostics to track newly emerging strains requires knowledge of antigenic and serological relationships of MERS-CoV to other coronaviruses.

Using synthetic genomics and a panel of Venezuelan equine encephalitis virus replicons (VRPs) expressing S and N proteins from MERS-CoV and other human and bat CoVs, the antigenic (using western blots and ELISA) and serological responses (using Neutralization Assays) against two MERS-CoV isolates were characterized in comparison with other human and bat coronaviruses.

Serologic and neutralization responses against the S glycoprotein were primarily strain specific with very low level cross reactivity within or across subgroups. N protein of coronaviruses within genoclusters, but not across subgroups, share cross-reactive epitopes with MERS-CoV isolates. These findings were validated using MERS-CoV patient (NA 01) convalescent serum and human serum to SARS-CoV, NL63 and OC43.

These studies identify a rapid response platform containing recombinant proteins and antisera that can be readily applied for detection, and control of MERS-CoV and other highly pathogenic zoonotic coronaviruses that may emerge in the future.

Vaccine design for emerging coronaviruses can involve chimeric S protein containing neutralizing epitopes from multiple virus strains across subgroups, to reduce immune pathology, and a diagnostic platform can include a panel of N and S proteins from phylogenetically distinct coronaviruses.

### Viruses, Cells and Plaque Assays.

MERS-CoV Hu/England-N1/2012 and MERS-CoV Hu/SA-N1/2012 were cultured on Vero 81 cells, grown in with Optimem (Gibco, CA) and 5% Fetal Clone Serum (Hyclone, South Logan, Utah) along with gentamycin/kanamycin (Gibco, Carlsbad, Calif.). Viral growth assays in Vero and Calu-3 cells were performed as previously described.

### Generation of Polyclonal Mouse Antisera, Neutralization Assays, Western and Northern Blot Analysis.

Genes encoding the indicated S and N proteins were synthesized from Bio BasicInc. (Ontario, Canada), and packaged into Venezuelan Equine Encephalitis virus replicon particles (VRPs) and mouse polyclonal sera were generated from BALB/c mice as described previously. Neutralization assays using mouse antisera involving MERS-CoV isolates and SARS-CoV were performed as described previously. For Western blots, lysates from cells infected with MERS-CoV isolates or VRPs expressing various S and N antigens were prepared as described before, and these blots were probed using the indicated mouse polyclonal sera. Vero cells inoculated with MERS-CoV isolates were harvested at 12HPI in Trizol Reagent (Invitrogen) were used to perform Northern blots.

### Enzyme Linked Immunosorbent Assay and Competition Assay.

ELISA analysis using indicated viruses or antigens expressed from VRPs was performed as described previously, and reactivity of mouse or human serum was determined using a chemiluminescence substrate. Competition assays were performed similarly with a mixture of mouse and patient serum.

### Molecular Characterization of MERS-CoV Hu/England-N1/2012.

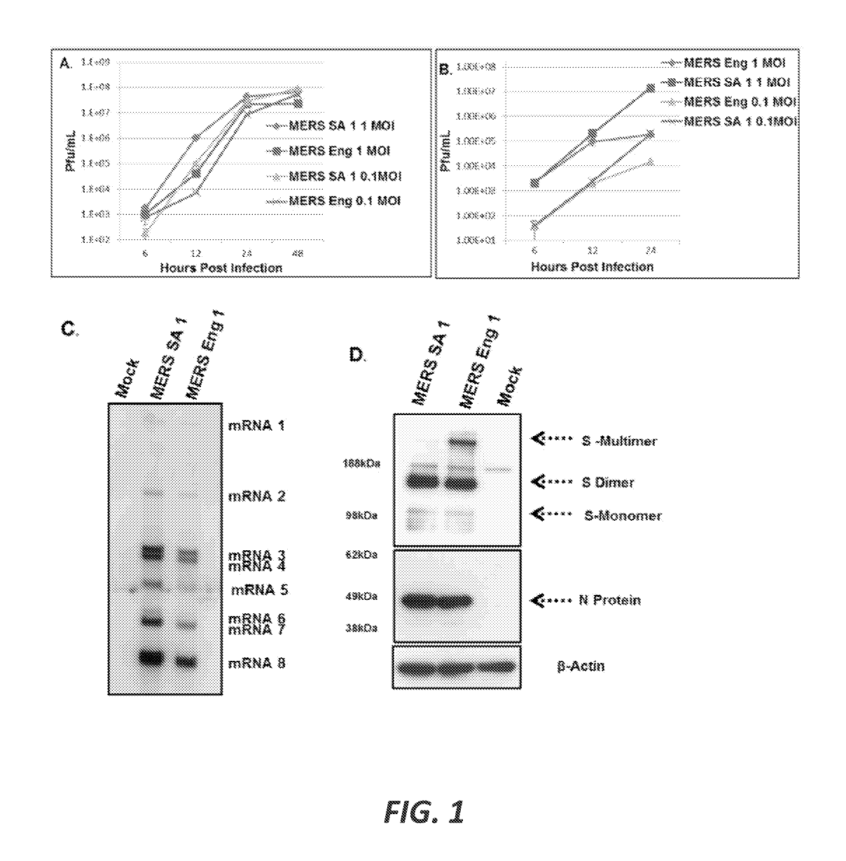

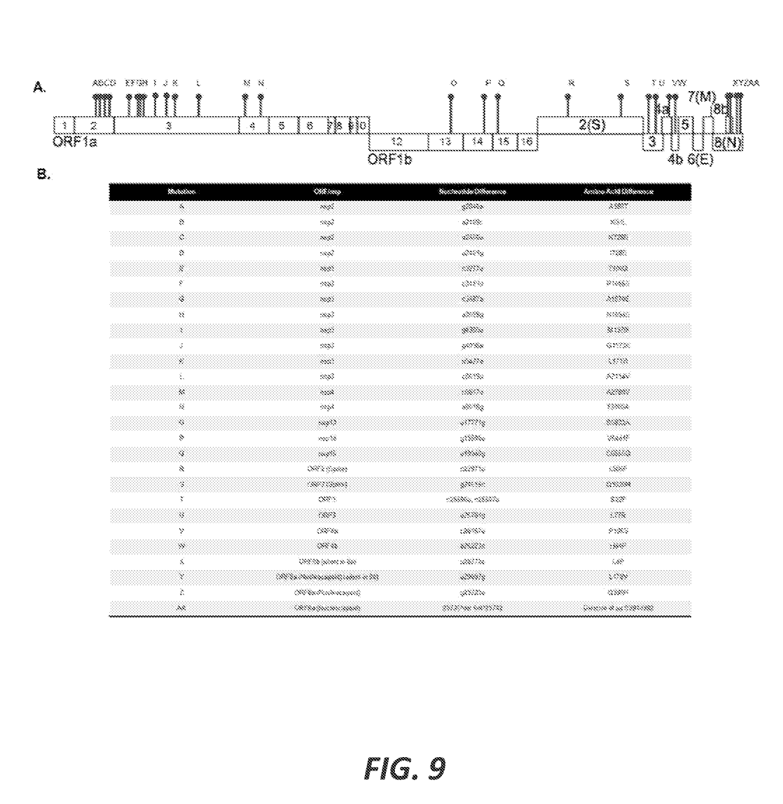

MERS-CoV Hu/England-N1/2012 (MERS Eng 1) was isolated from a 60 year old patient who exhibited severe respiratory illness and was transferred to London for treatment. Twenty nine mutations in MERS Eng 1 at the amino acid level were identified compared to published sequence of MERS-CoV Hu/SA-N1/2012 (MERS SA 1) (FIG. 9). To identify whether these mutations altered virus growth, the replication kinetics of the two isolates were analyzed in Vero cells and a continuous epithelial cell line, Calu-3 cells (FIGS. 1A-B). Although the replication kinetics was slightly different between two isolates in Vero cells, peak viral titers were equivalent. In contrast, virus growth was markedly distinct in human Calu-3 cells and could represent differences in strain specific in vitro adaptation phenotypes or result from functional differences in the sensitivity to innate immune responses.

Coronavirus replication and transcription involves production of nested set of sub genomic mRNAs, and previous reports predicted 10 open reading frames in MERS SA 1 and MERS Eng 1. Northern blot analysis identified eight sub genomic mRNAs after infection in both the viruses (FIG. 1C). The observed nested set of sub genomic mRNA expression is consistent with other coronaviruses. The predicted open reading frames (ORF) encoded by each mRNA (shown in Northern blots in FIG. 1C) in these MERS-CoV isolates is detailed in Table 1.

### Serologic Relationships Among MERS-CoV Strains

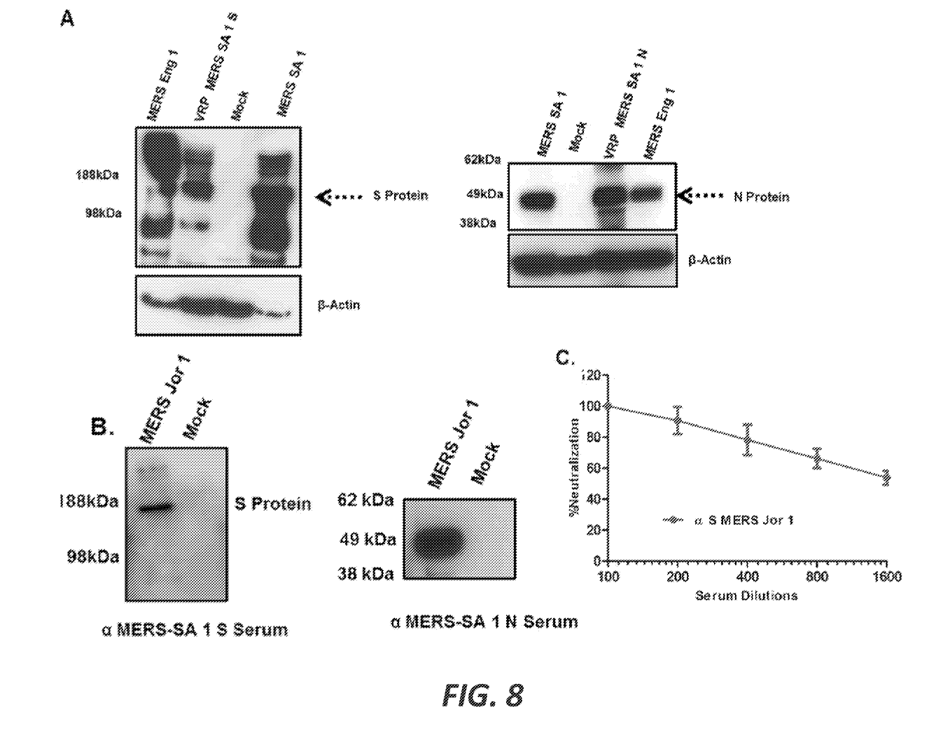

Venezuelan Equine Encephalitis Virus replicons (VRPs) function as efficient expression and vaccine delivery platforms for a variety of antigens. VRPs VRPs expressing MERS SA 1 S and N proteins were generated and used to immunize mice. N specific antiserum recognized a discrete 50 kDa band at the predicted molecular weight in lysates from Vero (FIG. 8A) and Calu-3 (FIG. 1D) cells infected with VRP-N or with the two different MERS-CoV isolates. For the most part, similar expression patterns were evident between VRPs and viruses; however, the N protein of MERS Eng 1 CoV had a slightly lower molecular weight, which was consistent with amino acid deletions at positions S391, I392 (FIG. 9).

Mouse anti-S serum identified three different molecular species of S protein in VRP-S or MERS-CoV infected Vero cells (FIG. 8A); S monomer (˜90 kDA), S dimer (˜180 kDa) and a larger multimer (>200 kDa). The observed molecular weights were consistent with the sizes of the S proteins of other CoVs. Similar results were noted in Calu-3 cells (FIG. 1D) and interestingly, the processing of S multimer in MERS Eng 1 appeared less efficient in both cell lines. Antiserum against MERS SA 1 S and N proteins also recognized MERS Eng 1 in ELISA assays (FIG. 8B).

### Cross Neutralization Patterns Across Strains.

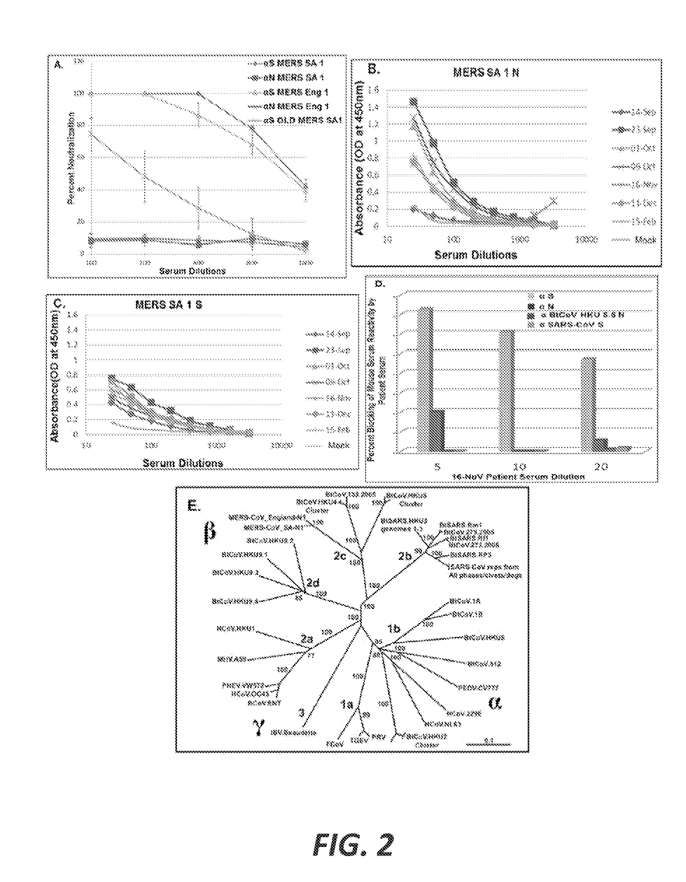

Plaque Reduction Neutralization Assays (PRNT₅₀) indicated complete neutralization of both MERS-CoV isolates by homotypic VRP-S antiserum (PRNT₅₀ titer: ˜1:1400 for both isolates) (FIG. 2A) and MERS-CoV Jordan isolate (FIG. 8C), whereas no neutralization was observed with N antiserum. Interestingly, serum from aged mice vaccinated with VRP S showed a six fold reduction in PRNT₅₀ titers (˜1:200), indicating that immunosenescnece plays a significant role in vaccine response to MERS CoV. Using serum from NA 01 patient, ELISA demonstrated high reactivity of the patient sera to N and S antigens of MERS SA 1 expressed from VRPs (FIGS. 2B-C). Patient sera collected early during infection showed high antibody titers to N protein that waned quickly over time, whereas sera collected at later time points showed high antibodies to the S glycoprotein. Most importantly, patient serum collected containing a high amount of antibodies to S protein outcompeted the binding of mouse S antiserum, to intact virus in a competition assay (FIG. 2D). These data suggest that similar/overlapping epitopes are recognized by human and mouse antisera following virus or VRP-S infection.

Cross Reactive and Cross Neutralizing Antibody Responses within and Across Alpha and Beta Coronaviruses.

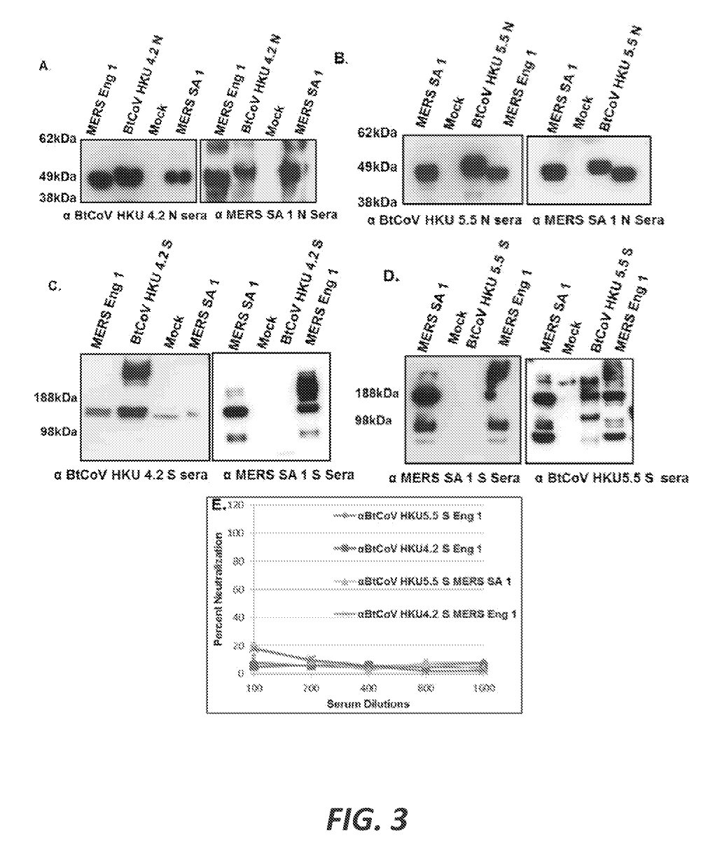

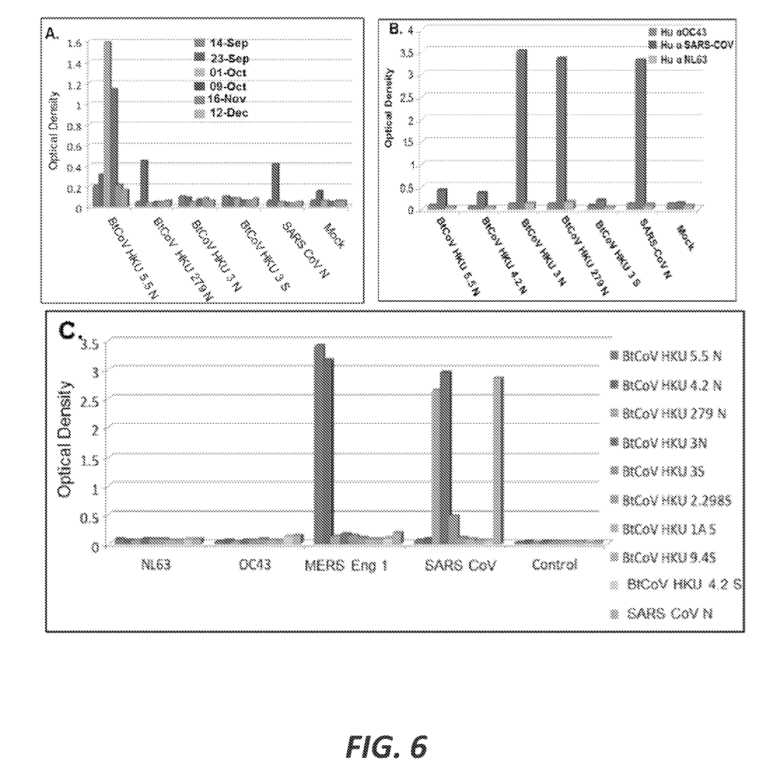

MERS SA 1 and MERS Eng 1 are closely related to BtCoV HKU5 and BtCoV HKU4 (FIG. 2E). To evaluate antigenic relationships with the group 2c betacoronaviruses, VRPs expressing S and N proteins of BtCoV HKU 4.2 and BtCoV HKU5.5 were inoculated into mice. Antisera against both HKU 4.2 and 5.5 N proteins recognized the N proteins of both MERS-CoV isolates, while MERS SA 1 N antisera also detected the VRP-expressed HKU 4.2 and 5.5 N proteins by Western Blot (FIGS. 3A-B). Similar results were obtained using ELISA and immunofluorescence assays. In contrast, there was little if any observable cross reactivity observed between MERS SA 1 S antisera with the VRP-expressed S proteins of HKU4.2 and HKU 5.5, while antisera to HKU 5.5, but not HKU4.2 S proteins weakly recognized the S proteins of both MERS-CoV isolates (FIGS. 3C-D). Serologic relationships were also measured using ELISA, which captures cross reactivity to conformational epitopes, and confirmed these antigenic relationships (FIG. 6C). Consistent with serology results, antisera against HKU4.2 and 5.5 S proteins did not cross neutralize the MERS-CoV isolates. These data indicate that the N, but not the S glycoprotein, are antigenically conserved within the Group 2c beta coronaviruses.

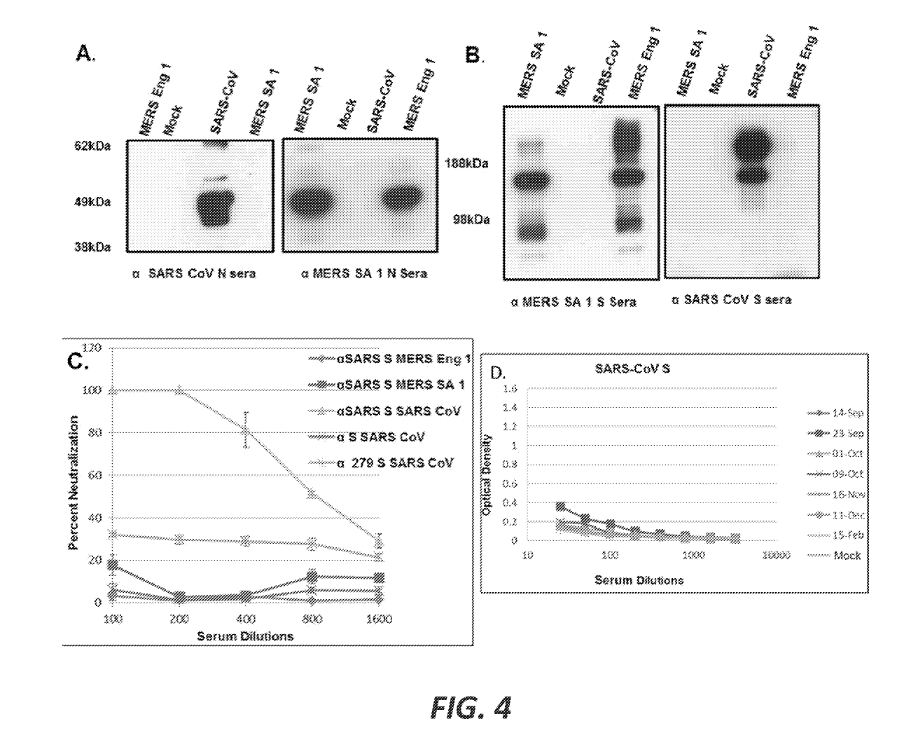

These analyses were extended to the highly pathogenic SARS-CoV and related group 2b betacoronaviruses. Polyclonal mouse sera to SARS-CoV or MERS SA 1 N or S proteins exhibited no cross reactivity to the reciprocal strains (FIG. 4A-B). Low levels of cross-neutralization of MERS SA 1 by mouse antisera to SARS-CoV were observed, using very high but not low concentrations of serum (FIG. 4C). Interestingly, ELISA results also showed very minimal cross-reactivity (FIG. 4D) of the NA 01 patient sera to SARS-CoV S antigen. Consistent with this observation, binding of mouse SARS-CoV S antiserum to SARS-CoV was not inhibited by NA01 patient sera in competition assays (FIG. 2D), indicating the absence of antibodies to SARS-CoV in the patient serum.

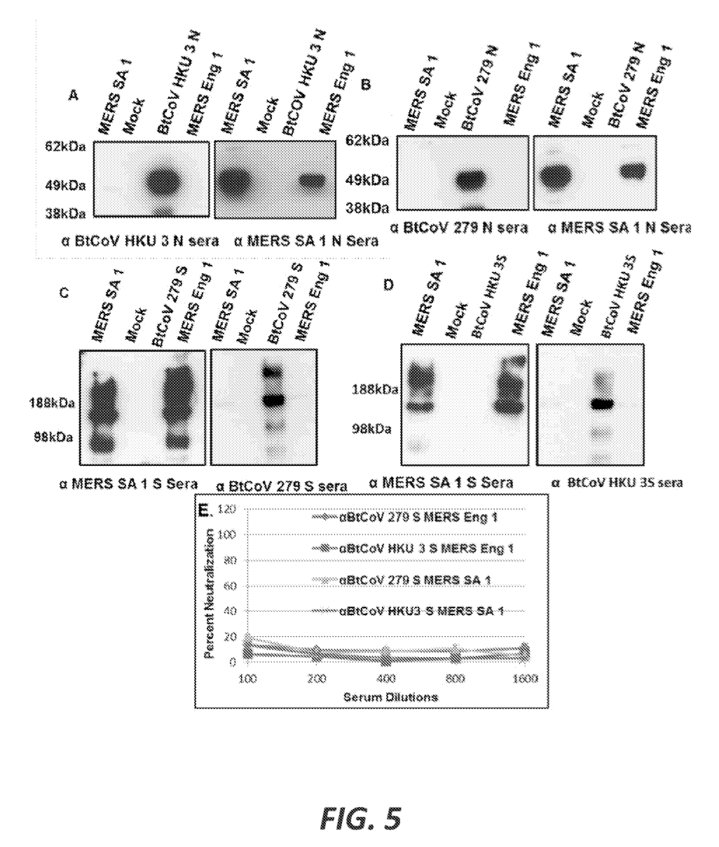

Consonant with these findings, no cross reactivity was observed with antisera against the VRP expressed N or S glycoproteins of BtCoV HKU3 and 279 and either MERS-CoV isolates (FIGS. 5A-D). Furthermore, no cross neutralization of MERS-CoV isolates by HKU 3S antiserum was observed. Interestingly, very low levels of cross neutralization of BtCoV 279 S antiserum against SARS-CoV (FIG. 4C) were observed.

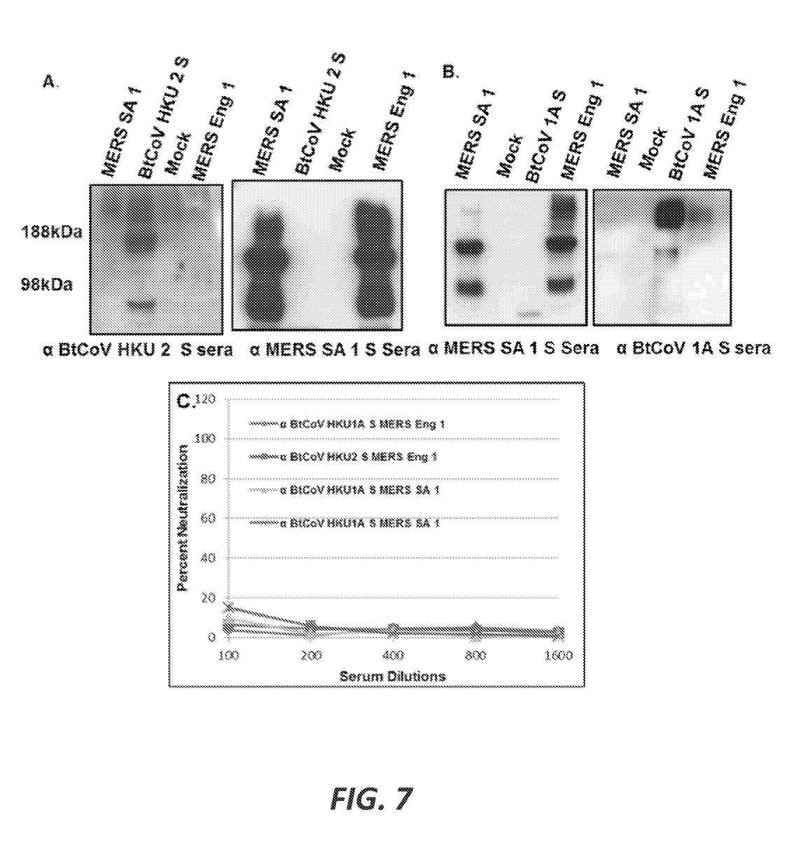

To further elucidate the antigenic relationships between the S glycoproteins of alpha coronaviruses to MERS-CoV isolates, mouse antisera to the BtCoV 1A and BtCoV HKU2 (alpha coronaviruses, Group 1b) were generated using the VRP platforms. Despite efficient recombinant S glycoprotein expression (FIGS. 7A-B), none of the recombinant S glycoproteins were recognized by MERS SA 1 S antisera. Antisera against BtCoV1A and HKU2 S glycoproteins had little if any cross reactivity with and did not neutralize MERS-CoV (FIGS. 7A-C).

### Antigenic Relationships Among the Human Coronaviruses

The antigenic relationships between VRP derived mouse serum with representative human coronaviruses from each subgroup (FIG. 6C); MERS Eng 1 (Group 2c), SARS-CoV (Group 2b), NL63 (Group 1b), and OC43 (Group 2a) were analyzed using ELISA. MERS Eng 1 was recognized by antisera targeting the N but not S glycoproteins of viruses within the group 2c betacoroanviruses. Likewise, SARS-CoV was only recognized by antisera to N, but not S glycoproteins of viruses with the group 2b betacoronaviruses. None of the antiserum screened reacted with other CoVs NL63 (Group 1b) or OC43 (Group 2a). Although BtCoV HKU2 is genetically close to NL63, no cross reactivity was observed within the S glycoprotein.

Serum from human patients infected with SARS-CoV, NL63 or OC43 was screened against the N proteins from representative subgroup 2c and 2d betacoronaviruses. Human serum to SARS-CoV recognized BtCoV HKU 3N, BtCoV 279N and SARS-CoV N (Group 2b), but did not recognize N proteins from other subgroups (FIG. 6B). Similarly, there was no cross reactivity of the human antisera from NL63 (Group 1 b) and OC 43 (Group 2a) infections with any of the viral antigens within the panel. Sera collected from the MERS-CoV NA01 patient showed cross-reactive binding only to BtCoV HKU 5.5 N (Group 2c), and little if any cross reactivity was noted outside the subgroup (FIG. 6A), except very low, transient cross detection of BtCoV 279 N, and SARS-CoV S and N recombinant proteins on a single day.

Using alphavirus replicon particles and synthetic gene design, a panel of recombinant proteins and sera from phylogenetically distant alpha and beta coronaviruses was assembled to evaluate the antigenic relationships between strains, and for informing vaccine design. MERS-CoV is a highly pathogenic respiratory coronavirus of humans, causing acute respiratory distress syndrome and mortality rates approaching 53%. Universal CoV primer sets were not successful in diagnosing the etiology of the Jordan outbreak in April 2012, demonstrating a critical need for paneled reagent sets of recombinant proteins and sera that allow for serologic evaluations of cases, contact cases and asymptomatic infections using Western blot or ELISA based techniques. These studies demonstrate the serological characterization of MERS-CoV and unrelated coronavirus diagnostic reagents. An advantage of the VRP platform is that it can also functions as a vaccine vector, affording the rapid production of candidate vaccines against newly emerged strains. Using SARS-CoV and MERS-CoV as models, these studies demonstrated that S protein based recombinant vaccines elicit robust neutralization responses in young and aged rodent models. As VRP-S vectors against SARS-CoV protected young and aged animals, a recombinant S vectored vaccine can be used that could likely prove successful in preventing heterologous MERS-CoV infection in the aged.

Two MERS-CoV isolates have been isolated to date which replicate well in Vero cells, but MERS Eng 1 replicated to lower titers in Calu-3 cells. As the two viruses have different passage histories in vitro, these differences may reflect the emergence of tissue culture adaptive mutations similar to observations reported with many SARS-CoV isolates. Alternatively, 29 amino acid differences have been described, most of which reside in the replicase polyprotein (FIG. 9). In addition, the S glycoprotein of MERS Eng 1 differs from MERS SA 1 by two amino acids L506F and Q1020H (FIG. 9), which may account for the increased amount of the higher molecular weight form of the S protein in MERS Eng 1 FIG. 1D, FIG. 8A). Also, a unique mutation T1015N has been identified in the MERS SA 1 isolate, but not in MERS Eng 1 and it has been shown that this mutation is responsible for increased in vitro fitness and plaque morphology. It is possible that the presence or absence of one or more of the S glycoprotein mutations in MERS Eng 1 may result in the slower growth phenotype in Calu3 cells.

Alphavirus VRPs are superior expression vectors with considerable potential as recombinant virus vaccine platforms and these studies demonstrate efficient expression of several coronavirus S and N structural proteins both in vitro and in vivo, resulting in robust serologic responses in vaccinated mice. Interestingly, antiserum to VRP-S glycoprotein but not to VRP-N protein neutralized both isolates of MERS-CoV, identifying the S glycoprotein as a principle target for neutralizing antisera. Furthermore, vaccine-induced immunopathology observed post challenge is not seen in VRP-S protein based vaccines, partly due to the Th1-biased immune response and high neutralization titers elicited by VRP vectors. The safety of VRP platforms has been demonstrated in high risk human populations and immunosenescent non-human primates and it is expected that these vectors will be efficacious in healthcare workers and target populations infected with MERS-CoV.

These results indicate the presence of strongly cross-reactive epitopes in the N protein within a particular genocluster, but not between genoclusters. Under identical conditions, little cross reactivity or conservation of cross-neutralizing epitopes was observed between S proteins within and across genoclusters. Importantly, the pattern of serologic and antigenic relationship observed using the mouse antisera was recapitulated using the human antiserum to four different CoVs. Neutralization assays demonstrated little if any conservation of cross neutralizing epitopes between S glycoproteins of CoVs within and across genoclusters. In particular, the absence of cross neutralization of MERS-CoV isolates by antiserum to HKU4 or HKU5 S glycoprotein, and SARS-CoV by antiserum to the HKU 3 or BtCoV 279 S glycoprotein suggests very limited conservation, or possibly, the deliberate masking of conserved cross-neutralizing sites within a genocluster. Although speculative, these cross neutralization relationships suggest that at least three antigenically distinct CoVs could emerge from zoonotic viruses circulating within group 1a/b, 2b and 2c reservoirs and then simultaneously circulate in humans. These findings argue that vaccine design for any new emerging coronavirus should either focus on the development of chimeric S glycoproteins containing neutralizing epitopes from multiple strains within or across genoclusters or develop new paradigms in structure-guided antigen design that improves the presentation of broadly neutralizing epitopes. Regions of S glycoprotein are interchangeable between coronaviruses within and across subgroups, rendering viable recombinant viruses. Inclusion of N protein in such chimeric vaccines may broaden the protective response. Such a vaccine might provide robust protection against several homologous and heterologous viruses within or across genoclusters.

After the SARS-CoV epidemic and in stark contrast to the situation with emerging influenza viruses like H7N9, the research and biomedical communities failed to develop broadly applicable biopreparedness platforms for rapid response against future emerging CoV threats. As CoVs have demonstrated an accelerating pattern of zoonoses since the 1980s, these data indicate that an appropriate diagnostic platform should include a large panel of phylogenetically distinct CoV S and N structural proteins, which must be validated using larger panels of antisera against other human coronaviruses and the general population. While molecular-based platforms like PCR and deep sequencing offer advantages in early detection of active infections, public health response platforms would be strengthened by the availability of recombinant proteins and group and type specific antisera that can track subclinical infections, determine the prevalence of infection in populations and identify hospital acquired infections. The VRP platform described herein not only provides for high level expression of key recombinant proteins across the alpha and beta CoVs, but provides the first candidate vaccine vectors with potential to augment Th1 based immune responses, and reduced immune pathology, for controlling MERS-CoV infection. The VRP 3526 approach is applicable to other highly pathogenic emerging infectious diseases for improving public health response, and control of future disease outbreaks in human populations. Finally, synthetic genome design provides a strategy to prepare recombinant viruses that allow for therapeutic evaluation and testing of antiviral compounds against future emerging CoVs.

The foregoing examples are illustrative of the present invention, and are not to be construed as limiting thereof. The invention is described by the following claims, with equivalents of the claims to be included therein.

All publications, patent applications, patents and other references cited herein are incorporated by reference in their entireties for the teachings relevant to the sentence and/or paragraph in which the reference is presented.

TABLE 1

| | | MERSCoVHu/ |
|---|---|---|
| mRNA | MERSCoVHu/SA-N1/2012 | England-N1/2012 |
| mRNA 1 | Full length Polyprotein | Full length Polyprotein |
| mRNA 2 | Spike, NS3a-d, E, M, N, 8b. | Spike, NS3a-d, E, M, N, 8b. |
| mRNA 3 | NS3a-d, E, M, N, 8b | NS3a-d, E, M, N, 8b |
| mRNA 4 | NS 3b-d, E, M, N, 8b | NS 3b-d, E, M, N, 8b |
| mRNA 5 | NS3d, E, M, N, 8b | NS3d, E, M, N, 8b |
| mRNA 6 | E, M, N, 8b | E, M, N, 8b |
| mRNA 7 | M, N, 8b | M, N, 8b |
| mRNA 6 | N, 8b | N, 8b |

1. A method of detecting the presence of a coronavirus in a sample and identifying the subgroup of the coronavirus in the sample, comprising:
a) contacting a sample with a panel of proteins comprising:
1) one or more nucleocapsid proteins from a subgroup 2c coronavirus,
2) one or more nucleocapsid proteins from a subgroup 2b coronavirus,
3) one or more nucleocapsid proteins from a subgroup 2a coronavirus,
4) one or more nucleocapsid proteins from a subgroup 2d coronavirus,
5) one or more nucleocapsid proteins from a subgroup 1a coronavirus,
6) one or more nucleocapsid proteins from a subgroup 1b coronavirus, and
7) any combination of (1) through (6) above,
under conditions whereby an antigen/antibody complex can form; and
b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b.

2. The method of claim 1, further comprising contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

3. A method of identifying a coronavirus spike protein for administration to elicit an immune response to coronavirus in a subject infected by a coronavirus and/or a subject at risk of coronavirus infection and/or to a subject for whom eliciting an immune response to a coronavirus is needed or desired, comprising:
a) contacting a sample obtained from a subject infected with a coronavirus with a panel of proteins comprising:
1) one or more spike proteins from a subgroup 2c coronavirus,
2) one or more spike proteins from a subgroup 2b coronavirus,
3) one or more spike proteins from a subgroup 2a coronavirus,
4) one or more spike proteins from a subgroup 2d coronavirus,
5) one or more spike proteins from a subgroup 1a coronavirus,
6) one or more spike proteins from a subgroup 1b coronavirus, and
7) any combination of (1) through (6) above,
under conditions whereby an antigen/antibody complex can form; and
b) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired.

4. The method of claim 3, further comprising contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired.

5. The method of claim 3, further comprising the step of administering the coronavirus spike protein identified according to the method to the subject of (a) and/or to a subject at risk of coronavirus infection and/or to a subject infected with a coronavirus and/or to a subject for whom eliciting an immune response to a coronavirus is needed or desired.

6. The method of claim 5, wherein the coronavirus spike protein is administered as part of an inactivated coronavirus.

7. The method of claim 5, wherein the coronavirus spike protein is administered as a VRP.

8. A method of identifying a coronavirus spike protein for administration to elicit an immune response to coronavirus in a subject infected with a coronavirus and/or to a subject at risk of coronavirus infection and/or to a subject for whom eliciting an immune response to coronavirus is needed or desired, comprising:
a) contacting a sample obtained from a subject known or suspected to be infected with a coronavirus with a panel of proteins comprising:
1) one or more nucleocapsid proteins from a subgroup 2c coronavirus,
2) one or more nucleocapsid proteins from a subgroup 2b coronavirus,
3) one or more nucleocapsid proteins from a subgroup 2a coronavirus,
4) one or more nucleocapsid proteins from a subgroup 2d coronavirus,
5) one or more nucleocapsid proteins from a subgroup 1a coronavirus,
6) one or more nucleocapsid proteins from a subgroup 1b coronavirus, and
7) any combination of (1) through (6) above,
under conditions whereby an antigen/antibody complex can form;
b) detecting formation of an antigen/antibody complex, whereby detection of formation of the antigen/antibody complex detects a coronavirus in the sample and whereby detection of formation of an antigen/antibody complex comprising the nucleocapsid protein(s) of (1) identifies the subgroup of the coronavirus in the sample as subgroup 2c; detection of an antigen/antibody complex comprising the nucleocapsid protein(s) of (2) identifies the subgroup of the coronavirus in the sample as subgroup 2b; detection of an antigen/antibody complex comprising the nucleocapsid protein(s) of (3) identifies the subgroup of the coronavirus in the sample as subgroup 2a; detection of an antigen/antibody complex comprising the nucleocapsid protein(s) of (4) identifies the subgroup of the coronavirus in the sample as subgroup 2d; detection of an antigen/antibody complex comprising the nucleocapsid protein(s) of (5) identifies the subgroup of the coronavirus in the sample as subgroup 1a; and detection of an antigen/antibody complex comprising the nucleocapsid protein(s) of (6) identifies the subgroup of the coronavirus in the sample as subgroup 1b;
c) contacting the sample from the subject with a panel of proteins comprising:
1) one or more spike proteins from a subgroup 2c coronavirus,
2) one or more spike proteins from a subgroup 2b coronavirus,
3) one or more spike proteins from a subgroup 2a coronavirus,
4) one or more spike proteins from a subgroup 2d coronavirus,
5) one or more spike proteins from a subgroup 1a coronavirus,
6) one or more spike proteins from a subgroup 1b coronavirus, and
7) any combination of (1) through (6) above,
under conditions whereby an antigen/antibody complex can form; and
d) detecting formation of an antigen/antibody complex, whereby detection of formation of the antigen/antibody complex comprising the spike protein(s) of any of (1)-(6) identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to coronavirus is needed or desired.

9. The method of claim 8, further comprising contacting the sample with one or more nucleocapsid proteins from a subgroup 3 coronavirus, whereby detection of an antigen/antibody complex comprising the nucleocapsid protein(s) of a subgroup 3 coronavirus identifies the subgroup of the coronavirus in the sample as a subgroup 3 coronavirus.

10. The method of claim 8, further comprising contacting the sample with one or more spike proteins from a subgroup 3 coronavirus, whereby detection of an antigen/antibody complex comprising the spike protein(s) of a subgroup 3 coronavirus identifies the presence of antibodies to a spike protein of the coronavirus that is infecting the subject of (a), thereby identifying a coronavirus spike protein for administration to the subject of (a) and/or to a subject infected with a coronavirus and/or to a subject at risk of coronavirus infection and/or to a subject for whom eliciting an immune response to a coronavirus is needed or desired.

11. The method of claim 8, further comprising the step of administering the coronavirus spike protein identified according to the methods and/or a coronavirus nucleocapsid protein of the coronavirus subgroup identified according to methods to the subject of (a) and/or to a subject infected with a coronavirus and/or to a subject at risk of coronavirus infection and/or to a subject for whom eliciting an immune response to a coronavirus is needed or desired.

12. The method of claim 11, wherein the coronavirus spike protein and/or is administered as part of an inactivated coronavirus.

13. The method of claim 11, wherein the coronavirus spike protein is administered as a VRP.

14. A method of identifying an antibody that neutralizes a coronavirus infecting a subject, comprising:
a) isolating a coronavirus from a sample of a subject infected with a coronavirus and/or suspected of being infected with a coronavirus;
b) contacting the coronavirus of (a) with a panel of antibodies comprising:
1) an antibody reactive with a spike protein from a subgroup 2c coronavirus,
2) an antibody reactive with a spike protein from a subgroup 2b coronavirus,
3) an antibody reactive with a spike protein from a subgroup 2a coronavirus,
4) an antibody reactive with a spike protein from a subgroup 2d coronavirus,
5) an antibody reactive with a spike protein from a subgroup 1a coronavirus,
6) an antibody reactive with a spike protein from a subgroup 1b coronavirus, and
7) any combination of (1) through (6) above,
to form respective coronavirus/antibody compositions, each comprising a respective antibody of the panel;
c) contacting each of the respective coronavirus/antibody compositions of (b) with cells susceptible to coronavirus infection under conditions whereby coronavirus infection can occur; and
d) detecting the presence or absence of infection of the cells, whereby absence of detection of infection of the cells contacted with any of the coronavirus/antibody compositions of (b) identifies the antibody of that coronavirus/antibody composition as an antibody that neutralizes the coronavirus infecting the subject.

15. The method of claim 14, further comprising contacting the coronavirus of (a) with an antibody reactive with a spike protein from a subgroup 3 coronavirus to form a coronavirus/antibody and contacting the coronavirus/antibody composition with cells susceptible to coronavirus infection under conditions whereby coronavirus infection can occur; and detecting the presence or absence of infection of the cells, whereby absence of detection of infection of the cells contacted with the coronavirus/antibody composition identifies the antibody of that coronavirus/antibody composition as an antibody that neutralizes the coronavirus infecting the subject.

16. The method of claim 14, further comprising the step of administering the antibody identified according to the methods to the subject of (a) and/or to a subject infected with a coronavirus and/or to a subject at risk of coronavirus infection and/or to a subject for whom eliciting an immune response to a coronavirus is needed or desired.

17. A kit comprising reagents and instructions for carrying out the method of claim 1.
